(19) United States
(12) Patent Application Publication
(10) Pub. No.: US 2002/0142374 A1
(43) Pub. Date: Oct. 3, 2002

(54) GENERATION OF MODIFIED MOLECULES WITH INCREASED SERUM HALF-LIVES

(76) Inventors: MICHAEL GALLO, SAN JOSE, CA (US); RICHARD JUNGHANS, BOSTON, MA (US); ORIT FOORD, FOSTER CITY, CA (US)

Correspondence Address: FISH & NEAVE, 1251 AVENUE OF THE AMERICAS, 50TH FLOOR, NEW YORK, NY, 10020-1105, US

(21) Appl. No.: 09/375,924

(22) Filed: Aug. 17, 1999

Related U.S. Application Data
(60) Provisional application No. 60/096,868, filed on Aug. 17, 1998.

(51) Int. Cl. C12P 21/06; C12P 21/04; C12P 21/08; C07K 16/00

(52) U.S. Cl. 435/69.1; 435/69.6; 530/387.3; 530/388.1; 530/388.23

(57) ABSTRACT

In accordance with the present invention, there are provided methods for the extension of serum half-lives of proteinaceous molecules, particularly antibody molecules, and compositions of molecules modified in accordance with the methods of the invention. In accordance with a first aspect of the present invention, there is provided a method of modifying the half-life of an antibody through providing an antibody containing an FcRn binding domain or the genes encoding such antibody and physically linking the antibody or the antibody as encoded to a second FcRn binding domain. In accordance with a second aspect of the present invention, there is provided a molecule that contains at least two distinct FcRn binding moieties.

### CROSS-REFERENCE TO RELATED APPLICATION

This application claims the benefit of U.S. Provisional Application No. 60/096,868, filed Aug. 17, 1998.

### FIELD OF THE INVENTION

In accordance with the present invention, there are provided methods for the extension of serum half-lives of proteinaceous molecules, particularly antibody molecules, and compositions of molecules modified in accordance with the methods of the invention.

### BACKGROUND OF THE TECHNOLOGY

Antibodies represent a substantial percentage, approximately 25%, of the biopharmaceuticals that are either entering phase III clinical trials or coming to market. Antibodies offer several unique features that make them very attractive as therapeutic reagents. In addition to extremely high specificity and high affinity to targets, antibodies, depending on their isotype, offer unique biological functions including complement fixation. Serum proteins, including antibodies are often rapidly degraded, or catabolized, in the body.

The kidney accounts for approximately 90% of catabolism of immunoglobulin fragments. Wochner et al. J. Exp. Med. 126:207 (1967). It has been shown that clearance of molecules is greatly reduced when the effective molecular size of the molecules exceed 70 kDa, the glomerular filtration cutoff size. Knauf et al. “Relationship of Effective Molecular Size to Systemic Clearance in Rats of Recombinant Interleukin-2 Chemically Modified with Water-soluble Polymers,” J. Biochem. 263:15064-15070 (1988). Nevertheless, antibodies of several gamma isotypes (IgGs), which have relative molecular sizes of approximately 150 kDa, uniquely possess relatively extended serum half-lives relative to other serum proteins (Humphrey and Fahey J. Clin. Invest. 40:1696-1705 (1961) and Sell J. Exp. Med. 120:967-986 (1966)).

In relation to the relatively extended half-life of IgG molecules, IgG molecules are protected from degradation by certain endosomal receptors that have been defined in recent studies (Junghans and Anderson PNAS USA 93:5512-5516 (1996)). Brambell et al. (Nature 203:1352-1355 (1964)) suggested that a specific receptor exists in rapid equilibrium with the intravascular space that protects IgG molecules from degradation. See also Brambell The Lancet ii:1087-1093 (1966). Significant work has been done to identify molecularly the region of the IgG molecule that binds to the receptor and understand the specific interaction between IgG molecules and their receptor (FcRb/FcRn) (Medesan et al. Eur. J. Immunol. 26:2533-2536 (1996); Vaughn and Bjorkman Structure 6:63-73 (1998); and Kim et al. Eur J. Immunol. 24:2429-2434 (1994)). The interaction of IgG with the FcRb receptor is pH dependent (binding at pH 6.0 and dissociating at pH 7.0) and has also been studied in some detail (Wallace and Rees Biochem. J. 188:9-16 (1980) and Raghaven Biochem. 34:14649-14657 (1995)). The presence of the Ig receptor suggests that specific sequences or conformations of an Ig molecule bind to the receptor. In support of this hypothesis, the same in vivo half-life has been observed for an Fc fragment containing the constant region derived from proteolysis of an IgG molecule and an intact IgG molecule, whereas Fab fragments (which do not contain the Fc domain) are rapidly degraded. Spiegelberg and Wiegle J. Exp. Med. 121:323-338 (1965); Waldmann and Ghetie “Catabolism of Immunoglobulins,” Progress in Immunol. 1:1187-1191 (Academic Press, New York: 1971); Spiegelberg in 19 Advances in Immunology F. J. Dixon and H. G. Kinkel, eds. 259-294 (Academic Press, NY: 1974); and Zuckier et al. Semin. Nucl. Med. 19:166-186 (1989) (review).

Further, it was generally believed that the relevant sequences leading to longer half-life of a murine IgG₂ molecule resided in the CH2 or CH3 domains and that deletion of one or the other domain would give rise to rapid degradation. An experiment analyzing the role of such domains demonstrated that a CH2 domain fragment, produced by trypsin digestion of the Fc region of a human IgG, persisted in the circulation of rabbits for as long as the intact Fc fragment or the intact IgG molecule from which such CH2 domain was produced. In contrast, an equivalent CH3 domain fragment, also produced by trypsin digestion of the Fc fragment, was rapidly eliminated, further supporting the hypothesis that an Ig receptor binding domain of IgG molecules resides in the CH2 domain of the molecule. Ellerson et al. J. Immunol. 116:510 (1976); Yasmeen et al. J. Immunol. 116:518 (1976). Yet other studies have shown that sequences in the CH3 domain are important in determining the different intravascular half-lives of IgG_{2b} and IgG₂ₐ antibodies in the mouse. Pollock et al. Eur. J. Immunol. 20:2021-2027 (1990).

Experiments have also been conducted that demonstrate that the rates of clearance of IgG variants that do not bind the FcRI or Clq receptors are the same as those for the parent wild-type antibody, indicating that the catabolic site is distinct from the sites involved in FcRI or Clq binding. Wawrzynczak et al. Molec. Immunol. 29:221 (1992). Removal of carbohydrate residues from IgG molecules or Fc fragments (though apparently dependent somewhat on the isotype of the molecule) has minimal to no effect on the in vivo half-life of the molecules. Nose and Wigzell Proc. Natl. Acad. Sci. USA 80:6632 (1983); Tao and Morrison J. Immunol. 143:2595 (1989); Wawrzynczak et al. Mol. Immunol. 29:213 (1992).

Clearance studies have been conducted in connection with Ig fusion or Ig complexed molecules. For example, Staphylococcal protein A (SpA)-IgG complexes were found to clear more rapidly from the serum than uncomplexed IgG molecules. Dima et al. Eur. J. Immunol. 13: 605 (1983). Site-directed mutagenesis studies have been conducted to determine if residues near the Fc-SpA interface are involved in IgG clearance. Kim et al. Eur. J. Immunol. 24:542-548 (1994). In such studies, amino acid residues of a recombinant Fc-hinge fragment derived from a murine IgG₁ molecule were changed and the effects of such mutations on the pharmacokinetics of the Fc-hinge fragment were determined. The study demonstrated that a site within the CH2-CH3 domain and overlapping with the SpA binding site of the molecule appeared to control the rate of catabolism. See also International Patent Application, WO 93/22332.

The role of concentration on catabolism is studied in Zuckier et al. Cancer 73:794-799 (1994). IgG catabolism is also discussed by Masson, J. Autoimmunity 6:683-689 (1993).

In view of the relatively extended half-life of IgG molecules as compared to other serum proteins, certain groups have attempted to either incorporate features of the IgG molecule in combination with other proteins, modify IgG molecules, or otherwise extend half-life of molecules based on the foregoing information. For example, International Patent Application No. 97/44362 (Anasetti et al.) discloses the generation of mutant IgG₂ molecules having extended serum half-lives. International Patent Application No. WO 97/43316 (Junghans) relates to the modification of molecules to enable Fc receptor binding in order to extend half-lives of the molecules. International Patent Application No. WO 97/34631 (Ward) discloses modified molecules having one or more amino acid substitutions in their Fc-hinge region such that antibody half-life is extended. International Patent Application No. WO 96/32478 (Presta and Snedecor) discloses modified molecules comprising a salvage receptor binding epitope of an Fc region of an IgG which have extended serum half-lives. International Patent Application No. WO 96/18412 discloses chimeric proteins bound to a polypeptide that comprises a lytic Fc fragment for extending serum half-life.

International Patent Application No. WO 96/08512 (Baker et al.) relates to altered Fc receptor-like polypeptides. International Patent Application No. WO 94/04689 (Pastan et al.) discloses a protein with a cytotoxic domain, a ligand-binding domain, and a peptide linking these two domains comprising an IgG constant region domain for the purpose of extending the half-life of the protein in vivo. In International Patent Application No. WO 93/22332 (Ward and Kim), the authors disclose a variety of experiments related to the mutation of CH2 and/or CH3 domains for enhancing stability and/or half-lives of molecules.

International Patent Application No. WO 91/08298 (Capon and Lasky) relates to fusion proteins bound preferably to Ig molecules for extending half-life of the molecule.

Indeed, the ability to prolong the serum half-life of antibodies would potentially reduce the costs of therapy, increase efficacy, and reduce toxicity.

### BRIEF DESCRIPTION OF THE DRAWING FIGURES

FIG. 1 is a schematic diagram of the design and construction of a modified molecule in accordance with the invention wherein the modified molecule is an antibody molecule conjugated to a hinge, CH2, and CH3 domain of an IgG FC region.

FIG. 2 is a schematic diagram of a method of a vector for the modification of an antibody with a second FcRn binding moiety in accordance with a preferred embodiment of the present invention.

FIG. 3 is a bar graph showing the competition between a modified molecule in accordance with the invention (clear bars) as compared to a wild type molecule (shaded bars).

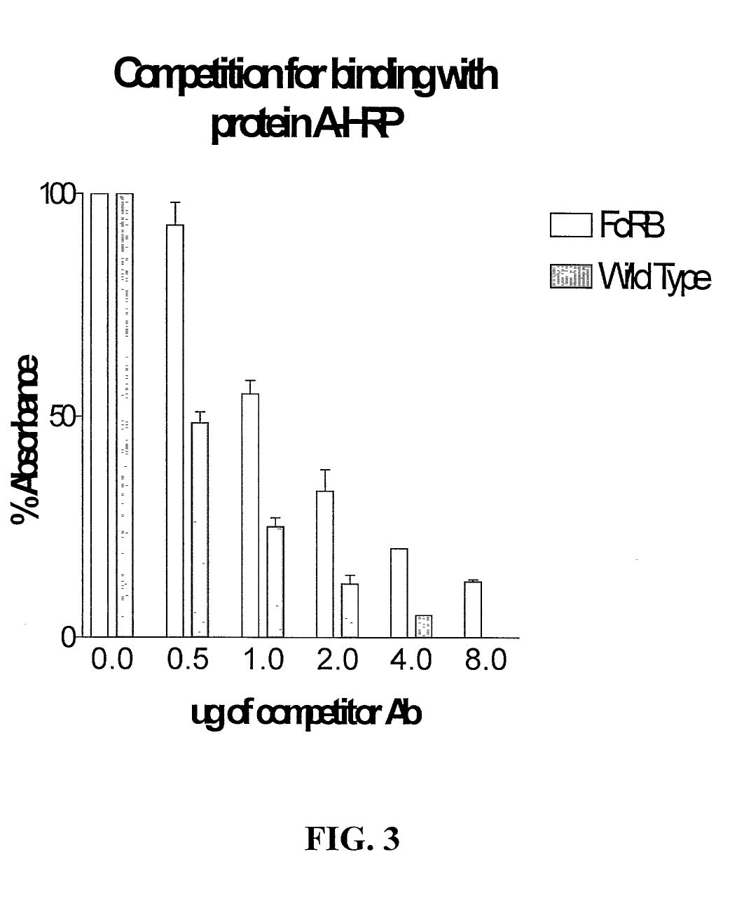

### SUMMARY OF THE INVENTION

In accordance with a first aspect of the present invention, there is provided a method of modifying the half-life of an antibody through providing an antibody containing an FcRn binding domain or the genes encoding such antibody and physically linking the antibody or the antibody as encoded to a second FcRn binding domain.

In accordance with a second aspect of the present invention, there is provided a molecule that contains at least two distinct FcRn binding moieties.

### DETAILED DESCRIPTION OF THE PREFERRED EMBODIMENTS

A. Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, tissue culture, and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g. free of murine proteins, (3) is expressed by a cell from a different species, or (4) does not occur in nature.

The term “polypeptide” is used herein as a generic term to refer to native protein, fragments, or analogs of a polypeptide sequence. Hence, native protein, fragments, and analogs are species of the polypeptide genus.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

The term “control sequence” as used herein refers to polynucleotide sequences which are necessary to effect the expression and processing of coding sequences to which they are ligated. The nature of such control sequences differs depending upon the host organism; in prokaryotes, such control sequences generally include promoter, ribosomal binding site, and transcription termination sequence; in eukaryotes, generally, such control sequences include promoters and transcription termination sequences. The term “control sequences” is intended to include, at a minimum, all components whose presence is essential for expression and processing, and can also include additional components whose presence is advantageous, for example, leader sequences and fusion partner sequences.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “oligonucleotide” referred to herein includes naturally occurring, and modified nucleotides linked together by naturally occurring, and non-naturally occurring oligonucleotide linkages. Oligonucleotides are a polynucleotide subset generally comprising a length of 200 bases or fewer. Preferably oligonucleotides are 10 to 60 bases in length and more preferably 12, 13, 14, 15, 16, 17, 18, 19, or 20 to 40 bases in length. Oligonucleotides are usually single stranded, e.g. for probes; although oligonucleotides may be double stranded, e.g. for use in the construction of a gene mutant. Oligonucleotides of the invention can be either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phoshoraniladate, phosphoroamidate, and the like. See e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990), the disclosures of which are hereby incorporated by reference. A oligonucleotide can include a label for detection, if desired.

The term “selectively hybridize” referred to herein means to detectably and specifically bind. Polynucleotides, oligonucleotides and fragments thereof in accordance with the invention selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, and fragments of the invention and a nucleic acid sequence of interest will be at least 80%, and more typically with preferably increasing homologies of at least 85%, 90%, 95%, 99%, and 100%. Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program. The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The following terms are used to describe the sequence relationships between two or more polynucleotide or amino acid sequences: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity. A “comparison window”, as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman Adv. Appl. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2^{nd} Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as a-, a-disubstituted amino acids, N-alkyl amino acids, lactic acid, and other unconventional amino acids may also be suitable components for polypeptides of the present invention. Examples of unconventional amino acids include: 4-hydroxyproline, g -carboxyglutamate, e-N,N,N-trimethyllysine, e-N-acetyllysine, O-phosphoserine, N-acetylserine, N-formylmethionine, 3-methylhistidine, 5-hydroxylysine, s-N-methylarginine, and other similar amino acids and imino acids (e.g., 4-hydroxyproline). In the polypeptide notation used herein, the lefthand direction is the amino terminal direction and the righthand direction is the carboxy-terminal direction, in accordance with standard usage and convention.

Similarly, unless specified otherwise, the lefthand end of single-stranded polynucleotide sequences is the 5′ end; the lefthand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity. Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, glutamic-aspartic, and asparagine-glutamine.

As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the invention.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W.H. Freeman and Company, New York (1984)); Introduction to Protein Structure (C. Branden and J. Tooze, eds., Garland Publishing, New York, N.Y. (1991)); and Thornton et at. Nature 354:105 (1991), which are each incorporated herein by reference.

The term “polypeptide fragment” as used herein refers to a polypeptide that has an amino-terminal and/or carboxy-terminal deletion, but where the remaining amino acid sequence is identical to the corresponding positions in the naturally-occurring sequence deduced, for example, from a full-length cDNA sequence. Fragments typically are at least 5, 6, 8 or 10 amino acids long, preferably at least 14 amino acids long, more preferably at least 20 amino acids long, usually at least 50 amino acids long, and even more preferably at least 70 amino acids long. The term “analog” as used herein refers to polypeptides which are comprised of a segment of at least 25 amino acids that has substantial identity to a portion of a deduced amino acid sequence and which desired biological function in vitro or in vivo. Typically, polypeptide analogs comprise a conservative amino acid substitution (or addition or deletion) with respect to the naturally-occurring sequence. Analogs typically are at least 20 amino acids long, preferably at least 50 amino acids long or longer, and can often be as long as a full-length naturally-occurring polypeptide.

Peptide analogs are commonly used in the pharmaceutical industry as non-peptide drus with properties analogous to those of the template peptide. These types of non-peptide compound are termed “peptide mimetics” or “peptidomimetics”. Fauchere, J. Adv. Drug Res. 15:29 (1986); Veber and Freidinger TINS p.392 (1985); and Evans et al. J. Med. Chem. 30:1229 (1987), which are incorporated herein by reference. Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from the group consisting of: —CH₂NH—, —CH₂S—, —CH₂—CH₂—, —CH═CH— (cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992), incorporated herein by reference); for example, by adding internal cysteine residues capable of forming intramolecular disulfide bridges which cyclize the peptide.

“Antibody” or “antibody peptide(s)” refer to an intact antibody, or a binding fragment thereof that competes with the intact antibody for specific binding. Binding fragments are produced by recombinant DNA techniques, or by enzymatic or chemical cleavage of intact antibodies. Binding fragments include Fab, Fab′, F(ab′)₂, Fv, and single-chain antibodies. An antibody other than a “bispecific” or “bifunctional” antibody is understood to have each of its binding sites identical. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay).

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody is said to specifically bind an antigen when the dissociation constant is £1 mM, preferably £ 100 nM and most preferably £ 10 nM.

The term “agent” is used herein to denote a chemical compound, a mixture of chemical compounds, a biological macromolecule, or an extract made from biological materials.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹¹I), fluorescent labels (e.g., FITC, rhodamine, lanthanide phosphors), enzymatic labels (e.g., horseradish peroxidase, b-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms or linkers of various lengths to reduce potential steric hindrance.

The term “pharmaceutical agent or drug” as used herein refers to a chemical compound or composition capable of inducing a desired therapeutic effect when properly administered to a patient. Other chemistry terms herein are used according to conventional usage in the art, as exemplified by The McGraw-Hill Dictionary of Chemical Terms (Parker, S., Ed., McGraw-Hill, San Francisco (1985)), incorporated herein by reference).

The term “antineoplastic agent” is used herein to refer to agents that have the functional property of inhibiting a development or progression of a neoplasm in a human, particularly a malignant (cancerous) lesion, such as a carcinoma, sarcoma, lymphoma, or leukemia. Inhibition of metastasis is frequently a property of antineoplastic agents.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term patient includes human and veterinary subjects.

B. Antibody Structure

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chain constant regions are classified as mu, delta, gamma, alpha, or epsilon, and define the antibody's isotype as IgM, IgD, IgG, IgA, and IgE, respectively. Each of the gamma heavy chain constant regions contain CH1, hinge, CH2, and CH3 domains, with the hinge domain in gamma-3 being encoded by 4 different exons. Morrison and Oi “Chimeric Ig Genes” in Immunoglobulin Genes pp. 259-274 (Honjo et al. eds., Academic Press Limited, San Diego, Calif. (1989)). Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site.

Thus, an intact antibody has two binding sites. Except in bifunctional or bispecific antibodies, the two binding sites are the same.

The chains all exhibit the same general structure of relatively conserved framework regions (FR) joined by three hyper variable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann Clin. Exp. Immunol. 79:315-321 (1990), Kostelny et al. J. Immunol. 148:1547-1553 (1992). Production of bispecific antibodies can be a relatively labor intensive process compared with production of conventional antibodies and yields and degree of purity are generally lower for bispecific antibodies. Bispecific antibodies do not exist in the form of fragments having a single binding site (e.g., Fab, Fab′, and Fv).

C. Introduction to the Present Invention

The present invention is specifically related to engineering of antibody molecules so as to contain a second IgG FcRn/FcRb binding domain in order to extend the serum half-life of such molecules and the characterization of these molecules in vitro and in vivo. However, as will be discussed herein, the present invention is also generally applicable to the extension of serum half-lives of a variety of molecules.

In accordance with the present invention there are provided methods for the utilization of a plurality of native or modified IgG CH domains to increase the avidity and/or affinity of the molecule incorporating the same with the FcRn receptor which is responsible for protecting IgG from catabolism. In this manner, serum half-lives of molecules modified in accordance with the invention can be extended. Also provided in accordance with the present invention are compositions of molecules modified in accordance with the methods of the invention. Generally, methods in accordance with the the invention comprise physically linking at least one molecule comprising an IgG CH like domain (a second FcRn binding moiety) to a molecule comprising an IgG CH like domain (a first FcRn binding moiety).

For example, an IgG antibody that ordinarily binds to FcRn represents a preferred first FcRn binding moiety and a molecule containing the CH2 and CH3 domains from an IgG Fc that ordinarily binds FcRn represents a second FcRn binding moiety. Physical linkage may be accomplished utilizing any conventional techniques. In preferred embodiments, physical linkage of the first and second FcRn binding moieties is accomplished recombinantly, i.e., wherein a gene construct encoding such first and second FcRn binding moieties are introduced into an expression system in a manner that allows correct assembly of the molecule upon expression therefrom. In this manner, where the first FcRn binding moiety is an IgG antibody that ordinarily binds to FcRn and the second FcRn binding moiety is a molecule containing the CH2 and CH3 domains from an IgG Fc that ordinarily binds FcRn, the molecule expressed may essentially been considered as an IgG antibody possessing a CH2 and CH3 domain dimer in its Fc region.

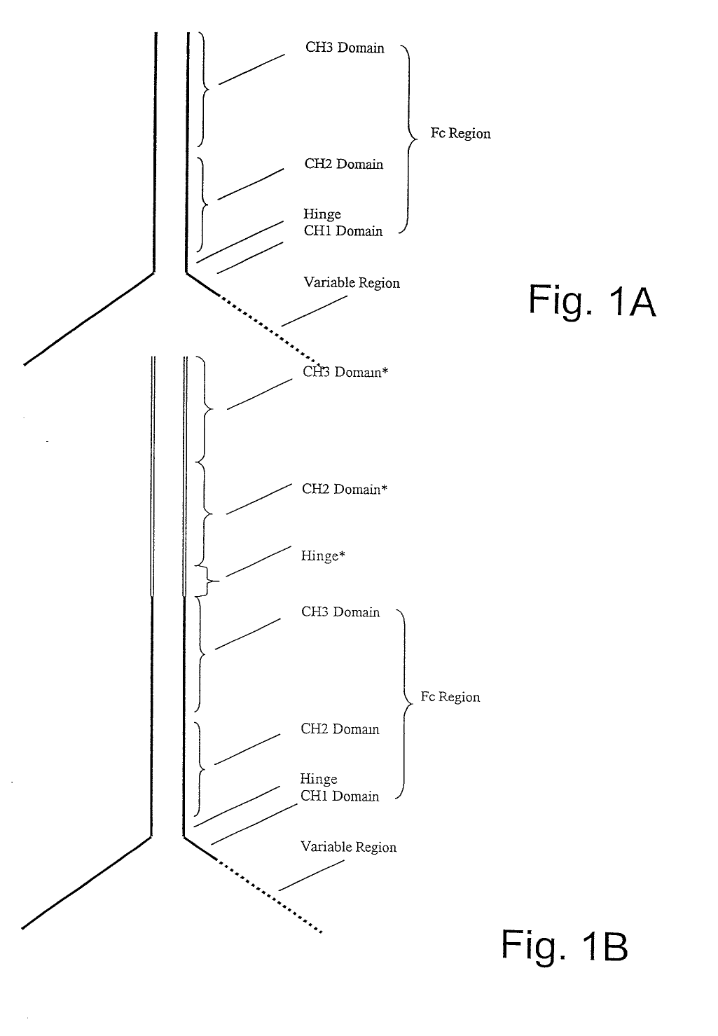

The foregoing example is depicted in FIGS. 1a and 1b. In FIG. 1a, an IgG antibody is pictorially represented showing the Fc region with its CH1, hinge, CH2, and CH3 domains. Such molecule represents a first FcRn binding moiety. In general, the genes encoding such molecule can be readily isolated and cloned into an expression system. Concurrently, or thereafter, the genes encoding a second FcRn binding moiety (i.e., the hinge, CH2, and CH3 domains from an Fc of an FcRn binding IgG antibody) can be isolated and cloned into the expression system. In this manner, the molecule depicted in FIG. 1b can be produced. Such molecule retains the structural elements of the first FcRn binding moiety (i.e., the Fc region with its CH1, hinge, CH2, and CH3 domains) and additionally acquires the structural elements introduced by the second FcRn binding moiety (i.e., the hinge*, CH2*, and CH3* domains).

Another manner in which to consider the present invention is in connection with the structure of the resulting molecule as modified in accordance with the present invention. From this perspective, compositions as modified in accordance with the present invention can be said to comprise at least two regions that bind to an FcRn. Such regions can be conceived as multimerized, though, the regions may be the same or may be different. As depicted in FIG. 1b, for example, the modified antibody presented possesses at least two regions that bind to FcRn through the presence of tandem CH2/CH3 domains derived from IgG Fc. In such a case, the regions are essentially the same. As will be appreciated, however, the regions might also be different and still convey to the molecule the property of possessing two regions that bind to an FcRn. One such example would be where the molecule is an antibody with a gamma-4 Fc that is engineered to possess the hinge, CH2, and CH3 domains from a gamma-1_Fc.

From the foregoing it will be understood by those in the art that the present invention can be utilized for increasing the serum half-life of many molecules. Moreover, the FcRn binding moiety need not be restricted to native forms of the FcRn binding moieties that are present in the Fc of IgG. Rather, FcRn binding moieties for use in accordance with the present invention can be generated through, for example, mutagenesis studies of Fc from IgG followed by screening for binding with FcRn (see e.g., Presta and Snedecor, U.S. Pat. No. 5,739,277) or peptide or polypeptide libraries can simply be screened for such binding. Such FcRn binding moieties, whether generated directly from Fc of IgG, derived from Fc of IgG and screened, or simply identified through screening, all may be useful in accordance with the present invention for extending serum half-lives of molecules, including antibody molecules, and in some cases may perform as well or better than Fc binding moieties generated directly from Fc of IgG.

The ability to significantly increase the serum half-life of antibody molecules, in particular, is highly advantageous. First, the longer serum half-life of an antibody would in all likelihood lower the amount of antibody needed in clinical treatments. The result could be significantly lower costs for treatment, since less material would be required. In addition, less frequent hospital visits due to fewer doses would increase the quality of life for patients, and potentially reduce the likelihood of toxicity. Second, extended antibody half-lives would also open the possibility of alternative routes of administration including intramuscular and subcutaneous administrations greatly increasing the general utility of antibodies as a therapeutic moiety. Third, as was already discussed above, the technology can potentially also be adapted to provide an extended serum half-life to other proteins in addition to antibodies. Nevertheless, these factors taken in combination, may increase the general utility of antibodies as a therapeutic moiety.

We believe that molecules in accordance with the present invention which possess at least 2 FcRn binding moieties will have greater avidity and/or affinity for the FcRn and FcRb receptors. We further expect that the presence of two or more receptor binding domains will act to alter the kinetics of receptor binding. Enhanced avidity/affinity is important since the FcRn/FcRb receptor is limiting in the endosome; only a small fraction of IgG molecules are rescued from catabolism (Junghans Immunologic Res. 16:29-57 (1997)). Thus, molecules in accordance with the present invention, if capable of out-competing normal IgG for binding to the FcRn/FcRb receptor, then we expect that the half-life of the molecules will be substantially increased. Such modified molecules are expected to still bind in a pH dependent and biologically relevant manner (pH 6.0). Moreover, in molecules where the receptor binding domain itself remains unmodified, the ability of the modified molecule to dissociate from the receptor at neutral pH, which is essential for recycling the antibody back to the plasma, should not be compromised.

It will be apprciated that the present invention is also applicable to enhancing the interactions between a receptor and its ligand generally. In this respect, either receptor or ligand moieties may be modified so as to generate molecules that possess greater than one moiety that enhances the affinity, avidity, or simply the ability of receptor and ligand to interact. Stated another way, the invention, by increasing the number of specific binding domains (doubling, tripling etc) provides a method to increase avidity of a molecule to its target. The end result is that the modified molecule will have a higher affinity for the target the parent molecule and consequently can be used as a competitor. In addition, because the modification does not introduce new protein sequences the modified molecules are less likely to be immunogenic. Below are several examples in which one of ordinary skill in the art would foresee the desire to generate such reagents.

One example would be the generation of a reagent or drug that would be able to bind to a virus/drug/toxin to prevent its binding to its natural receptor. Currently soluble receptors are being examined for their utility in a number of therapeutic situations. We believe that soluble receptor reagents could have greater utility if the receptors were constructed as multimers such that their affinities will be enhanced in accordance with the present invention. Adding additional binding domains should provide significant enhancement in avidity to out-compete the endogenous receptor. Again, since no additional sequences are introduced the immunogenicity should not be altered significantly.

Other ligand receptor interactions are also amendable to this strategy. Cell surface receptors including channel linked, g-protein-linked, and catalytic receptors all interact with specific ligands. In this case introducing multiple receptor binding domains a ligand molecule with higher affinities than the endogenous ligand can be generated. The ligand with higher affinity could be designed to block the function of the receptor as an antagonist or to potentially generate an extremely potent agonist. Linking a toxin might also provided a useful therapeutic. The method is applicable to both b adrenergic receptors that activate adenylate cyclase and a2 adrenergic receptors that inhibit adenylate cyclase. Of course as in the viral example above a soluble receptor that had been modified with multiple ligand binding sites would also yield a potentially useful reagent. Because the modified-soluble receptor would be capable of binding the ligand with high affinities (presumably both on rates and off rates would increase) it could be used to prevent the binding of a ligand to its receptor. This general approach can be applied to inhibiting the binding of virtually every cytokine or chemokine to its receptor and would be an improvement of current soluble receptor strategies. Cell-cell interactions and cell adhesion could clearly be disrupted or modified with molecules engineered with multiple binding domains. In fact, one can potentially imagine disrupting fertilization (sperm-egg adhesion) by engineering a very high affinity molecule comprising multiple binding domains for the human egg.

The invention has general utility for being exploited in any system that involves protein interactions including multi-enzyme complexes and allosteric proteins. Again the increased affinity provided by increasing the number of binding domains could be used to generate potent inhibitors that interfere with normal interactions. Potentially, modified proteins with increased number of specific binding domains could also yield more stable complexes or potent effector molecules. By generating molecules with multiple domains capable of binding signal peptide sequences or nuclear import signal sequences it is possible to improve the efficiency of these process or to generate potent antagonists to these processes.

Other biological systems including endocrine, paracrine and synaptic systems by virtue of utilizing specific receptor ligand binding could all be potentially manipulated with a modified molecule with multiple ligand/receptor binding sites. Steroid hormones or synthetic hormones may be improved by increasing the number of binding domains. Ligands do not have to be proteins, even calmodulin which is an ubiquitous intracellular receptor for Ca²⁺ could be potentially modified to yield a molecule with increase affinity for Ca²⁺. Carrier and channel proteins that transport sugars or amino acids can also be modified to yield molecules with high affinities for their respective ligands. Utility for the invention may also be found in manipulating lectin binding domains.

The invention, because it provides increase affinity between two molecules, could also be used in the design of more effective and powerful molecular reagents. By generating a modified-ligand with multiple binding domains for its receptor could provide dramatic increases in affinity to allow previously low affinity interactions to be probed for molecular studies.

D. Preparation of Antibodies

In preferred embodiments, where antibodies are utilized in accordance with the present invention, such antibodies are preferably humanized or human antibodies. A preferred method for the generation of human antibodies is through the use of generation of such antibodies in transgenic mammals. The ability to clone and reconstruct megabase-sized human loci in YACs and to introduce them into the mouse germline provides a powerful approach to elucidating the functional components of very large or crudely mapped loci as well as generating useful models of human disease. Furthermore, the utilization of such technology for substitution of mouse loci with their human equivalents could provide unique insights into the expression and regulation of human gene products during development, their communication with other systems, and their involvement in disease induction and progression.

An important practical application of such a strategy is the “humanization” of the mouse humoral immune system. Introduction of human immunoglobulin (Ig) loci into mice in which the endogenous Ig genes have been inactivated offers the opportunity to study the mechanisms underlying programmed expression and assembly of antibodies as well as their role in B-cell development. Furthermore, such a strategy could provide an ideal source for production of fully human monoclonal antibodies (Mabs)—an important milestone towards fulfilling the promise of antibody therapy in human disease. Fully human antibodies are expected to minimize the immunogenic and allergic responses intrinsic to mouse or mouse-derivatized Mabs and thus to increase the efficacy and safety of the administered antibodies. The use of fully human antibodies can be expected to provide a substantial advantage in the treatment of chronic and recurring human diseases, such as inflammation, autoimmunity, and cancer, which require repeated antibody administrations.

One approach towards this goal was to engineer mouse strains deficient in mouse antibody production with large fragments of the human Ig loci in anticipation that such mice would produce a large repertoire of human antibodies in the absence of mouse antibodies. Large human Ig fragments would preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains should yield high affinity antibodies against any antigen of interest, including human antigens. Using the hybridoma technology, antigen-specific human Mabs with the desired specificity could be readily produced and selected.

This general strategy was demonstrated in connection with our generation of the first XenoMouseÔ strains as published in 1994. See Green et al. Nature Genetics 7:13-21 (1994). The XenoMouseÔ strains were engineered with yeast artificial chromosomes (YACs) containing 245 kb and 190 kb-sized germline configuration fragments of the human heavy chain locus and kappa light chain locus, respectively, which contained core variable and constant region sequences. Id. The human Ig containing YACs proved to be compatible with the mouse system for both rearrangement and expression of antibodies and were capable of substituting for the inactivated mouse Ig genes. This was demonstrated by their ability to induce B-cell development, to produce an adult-like human repertoire of fully human antibodies, and to generate antigen-specific human Mabs. These results also suggested that introduction of larger portions of the human Ig loci containing greater numbers of V genes, additional regulatory elements, and human Ig constant regions might recapitulate substantially the full repertoire that is characteristic of the human humoral response to infection and immunization. The work of Green et al. was recently extended to the introduction of greater than approximately 80% of the human antibody repertoire through introduction of megabase sized, germline configuration YAC fragments of the human heavy chain loci and kappa light chain loci, respectively. See Mendez et al. Nature Genetics 15:146-156 (1997) and U.S. patent application Ser. No. 08/759,620, filed Dec. 3, 1996, the disclosures of which are hereby incorporated by reference.

Such approach is further discussed and delineated in U.S. patent application Ser. No. 07/466,008, filed Jan. 12, 1990, Ser. No. 07/610,515, filed Nov. 8, 1990, Ser. No. 07/919,297, filed Jul. 24, 1992, Ser. No. 07/922,649, filed Jul. 30, 1992, filed Ser. No. 08/031,801, filed Mar. 15,1993, Ser. No. 08/112,848, filed Aug. 27, 1993, Ser. No. 08/234,145, filed Apr. 28, 1994, Ser. No. 08/376,279, filed Jan. 20, 1995, Ser. No. 08/430,938, Apr. 27, 1995, Ser. No. 08/464,584, filed Jun. 5, 1995, Ser. No. 08/464,582, filed Jun. 5, 1995, Ser. No. 08/463,191, filed Jun. 5, 1995, Ser. No. 08/462,837, filed Jun. 5, 1995, Ser. No. 08/486,853, filed Jun. 5, 1995, Ser. No. 08/486,857, filed Jun. 5, 1995, Ser. No. 08/486,859, filed Jun. 5, 1995, Ser. No. 08/462,513, filed Jun. 5, 1995, Ser. No. 08/724,752, filed Oct. 2, 1996, and Ser. No. 08/759,620, filed Dec. 3, 1996. See also Mendez et al. Nature Genetics 15:146-156 (1997). See also European Patent No., EP 0 463 151 B1, grant published Jun. 12, 1996, International Patent Application No., WO 94/02602, published Feb. 3, 1994, International Patent Application No., WO 96/34096, published Oct. 31, 1996, PCT Application No. PCT/US96/05928, filed Apr. 29, 1996, and International Patent Application No. WO 98/24893, published Jun. 11, 1998. The disclosures of each of the above-cited patents, applications, and references are hereby incorporated by reference in their entirety.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more V_{H} genes, one or more D_{H} genes, one or more J_{H} genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806 and 5,625,825, both to Lonberg and Kay, and GenPharm International U.S. patent application Ser. No. 07/574,748, filed Aug. 29, 1990, Ser. No. 07/575,962, filed Aug. 31, 1990, Ser. No. 07/810,279, filed Dec. 17, 1991, Ser. No. 07/853,408, filed Mar. 18, 1992, Ser. No. 07/904,068, filed Jun. 23, 1992, Ser. No. 07/990,860, filed Dec. 16, 1992, Ser. No. 08/053,131, filed Apr. 26, 1993, Ser. No. 08/096,762, filed Jul. 22, 1993, Ser. No. 08/155,301, filed Nov. 18, 1993, Ser. No. 08/161,739, filed Dec. 3, 1993, Ser. No. 08/165,699, filed Dec. 10, 1993, Ser. No. 08/209,741, filed Mar. 9, 1994, the disclosures of which are hereby incorporated by reference. See also International Patent Application Nos. WO 94/25585, published Nov. 10, 1994, WO 93/12227, published Jun. 24, 1993, WO 92/22645, published Dec. 23, 1992, WO 92/03918, published Mar. 19, 1992, and WO 98/24884, published Jun. 11, 1998, the disclosures of which are hereby incorporated by reference in their entirety. See further Taylor et al., 1992, Chen et al., 1993, Tuaillon et al., 1993, Choi et al., 1993, Lonberg et al., (1994), Taylor et al., (1994), and Tuaillon et al., (1995), the disclosures of which are hereby incorporated by reference in their entirety.

The inventors of Surani et al., cited above and assigned to the Medical Research Counsel (the “MRC”), produced a transgenic mouse possessing an Ig locus through use of the minilocus approach. The inventors on the GenPharm International work, cited above, Lonberg and Kay, following the lead of the present inventors, proposed inactivation of the endogenous mouse Ig locus coupled with substantial duplication of the Surani et al. work.

An advantage of the minilocus approach is the rapidity with which constructs including portions of the Ig locus can be generated and introduced into animals. Commensurately, however, a significant disadvantage of the minilocus approach is that, in theory, insufficient diversity is introduced through the inclusion of small numbers of V, D, and J genes. Indeed, the published work appears to support this concern. B-cell development and antibody production of animals produced through use of the minilocus approach appear stunted. Therefore, research surrounding the present invention has consistently been directed towards the introduction of large portions of the Ig locus in order to achieve greater diversity and in an effort to reconstitute the immune repertoire of the animals.

Human anti-mouse antibody (HAMA) responses have led the industry to prepare chimeric or otherwise humanized antibodies. Certain antibodies have been prepared which are chimeric antibodies, having a human constant region and a murine variable region, it is expected that certain human anti-chimeric antibody (HACA) responses will be observed, particularly in chronic or multi-dose utilizations of the antibody.

Antibodies in accordance with the invention are preferably prepared through the utilization of a transgenic mouse that has a substantial portion of the human antibody producing genome inserted but that is rendered deficient in the production of endogenous, murine, antibodies. Such mice, then, are capable of producing human immunoglobulin molecules and antibodies and are deficient in the production of murine immunoglobulin molecules and antibodies. Technologies utilized for achieving the same are disclosed in the patents, applications, and references disclosed in the Background, herein. In particular, however, a preferred embodiment of transgenic production of mice and antibodies therefrom is disclosed in U.S. patent application Ser. No. 08/759,620, filed Dec. 3, 1996, the disclosure of which is hereby incorporated by reference. See also Mendez et al. Nature Genetics 15:146-156 (1997), the disclosure of which is hereby incorporated by reference.

Through use of such technology, we have produced fully human monoclonal antibodies to a variety of antigens. Essentially, we immunize XenoMouseÔ lines of mice (referred to herein as XenoMouse animals) with an antigen of interest, recover lymphatic cells (such as B-cells) from the mice that express antibodies, fuse such recovered cells with a myeloid-type cell line to prepare immortal hybridoma cell lines, and such hybridoma cell lines are screened and selected to identify hybridoma cell lines that produce antibodies specific to the antigen of interest. Such techniques have been utilized in accordance with the present invention for the preparation of antibodies and the like. In general, antibodies in accordance with the invention possess very high affinities, typically possessing Kd's of from about 10⁻⁹ through about 10⁻¹¹ M, when measured by either solid phase and solution phase.

As will be appreciated, antibodies in accordance with the present invention can be expressed in cell lines other than hybridoma cell lines. Sequences encoding particular antibodies can be used for transformation of a suitable mammalian host cell. Transformation can be by any known method for introducing polynucleotides into a host cell, including, for example packaging the polynucleotide in a virus (or into a viral vector) and transducing a host cell with the virus (or vector) or by transfection procedures known in the art, as exemplified by U.S. Pat. Nos. 4,399,216, 4,912,040, 4,740,461, and 4,959,455 (which patents are hereby incorporated herein by reference). The transformation procedure used depends upon the host to be transformed. Methods for introduction of heterologous polynucleotides into mammalian cells are well known in the art and include dextran-mediated transfection, calcium phosphate precipitation, polybrene mediated transfection, protoplast fusion, electroporation, encapsulation of the polynucleotide(s) in liposomes, and direct microinjection of the DNA into nuclei.

Mammalian cell lines available as hosts for expression are well known in the art and include many immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), and a number of other cell lines. Cell lines of particular preference are selected through determining which cell lines have high expression levels and produce antibodies with constitutive binding properties.

E. Construction of Modified Antibodies

As discussed above, a preferred modified molecule in accordance with the present invention is an antibody. The basic design used to that end is to incorporate a second FcRn binding domain onto the antibody. Published work has identified the IgG domains that bind to the FcRb receptor as being located at the CH2 and CH3 junction of the IgG molecule (Medesan et al. Eur. J. Immunol. 26:2533-2536 (1996); Vaughn and Bjorkman Structure 6:63-73 (1998); and Kim et al. Eur J. Immunol. 24:2429-2434 (1994)). One construct in accordance with the invention is the simple addition of a second CH2-CH3 domain to an existing antibody (as shown in FIG. 1b). In one embodiment, the “parent antibody” that we chose to modify is a human monoclonal antibody that was generated through immunization of a transgenic mouse, as described above, and is specific to the cytokine IL-8 and possesses an IgG4 isotype. Such antibody, thus, comprises a first FcRn binding moiety in connection with its gamma-4 Fc. We modified the antibody at the carboxy terminus of the constant region so that there would be no impact on the variable regions or the complementary determining regions (CDRs) which are responsible for antibody binding.

The most significant issue in the design of the modified antibody is the nature of the junction between the original CH3 domain of the antibody and the second FcRn binding moiety. We therefore, in one embodiment of the invention, utilized the hinge domain of the constant region as a linker. The hinge is flexible and assists in maintaining the natural structure of the antibody. The resulting construct thus contains an additional 26 kd representing the hinge-CH2-CH3 (see FIG. 1b and below). An additional advantage of this design is that the new molecule is not likely to be immunogenic.

The amino acid composition and length of the linker to separate the parent antibody immunoglobulin molecule from the second FcRn binding moiety is unknown. However, as will be appreciated, testing constructs containing a variety of different sequences is relatively simple. For example, we are cloning three different linkers, based on the hinge regions from three different IgG isotypes (IgG1, IgG2, and IgG4) utilizing strategies described herein and generating cell lines expressing the modified antibody with different linkers. In the Examples described below, we describe our work in connection with the gamma-1 hinge region as a linker.

As will be appreciated, where a modified molecule is prepared with a hinge region and depending upon the particular hinge region that is chosen, it may be preferable or necessary to introduce certain mutations so as to modify its interaction. Although a generic linker could be generated, we were interested in staying with Ig hinge regions for two reasons. First, the IgG hinge region in the native molecule serves the specific function to separate the Fab (VH+CH1 and light chain) from the CH2 and CH3 domains as a discrete entity (protease digestion releases the Fab). Secondly, we were interested in modifying molecules with predominantly human components such that the resulting molecules are as close to human as possible, or at least possess human-like junctions and sequences. Accordingly, we were interested in introducing as few amino-acid changes to the modified molecules as possible so as to avoid generating immunogenicity. Certain literature has suggested that the hinge region may be important for proper folding of the Ig molecule. Kim et al. Mol. Immunol. 32:467-475 (1995). Thus, in a preferred embodiment of the invention, we utilize native hinge region sequences in order to achieve more natural molecular conformations. The rest of the molecule, the FcRb binding domain comprising the CH2-CH3 domains, represents a tandem repeat or multimer of a portion of the parent Ig molecule and, thus, should not be immunogenic.

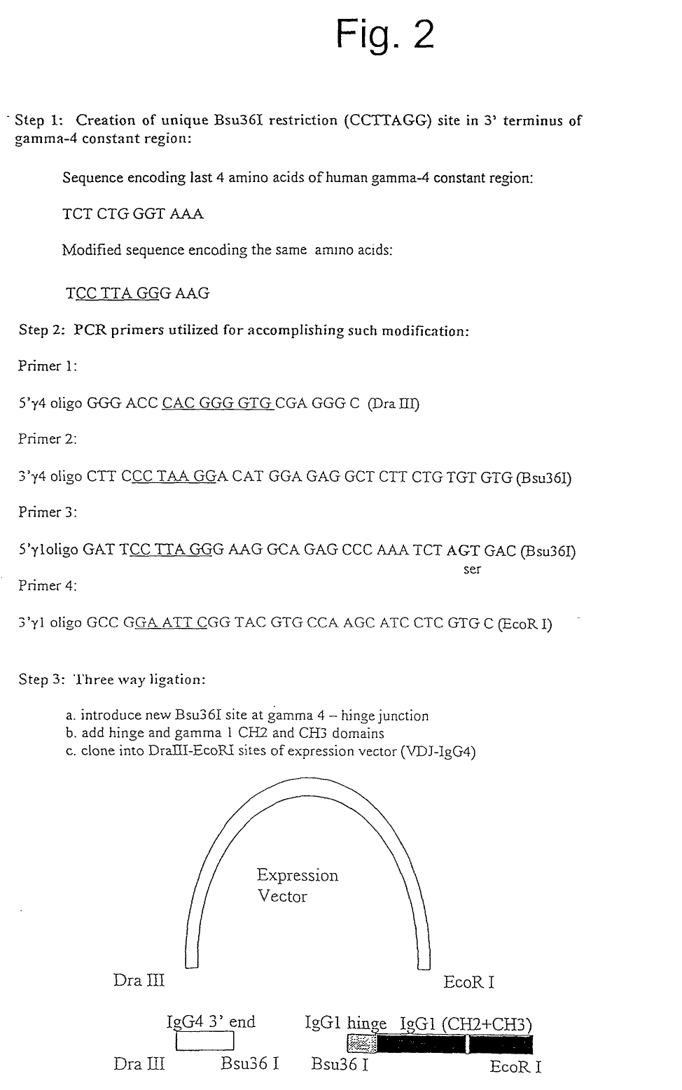

All IgG hinge regions contain cysteines that participate in interhinge linkage. The difference among the three isotypes, however, includes the distance between the beginning of the hinge and the first cysteine (3 amino acids for IgG2, 8 amino acids for IgG4 and 11 amino acids in the mutated IgG1; see FIG. 2). For example, where the gamma-1 hinge region is utilized, it is preferable to remove the cysteine, through mutation, that would normally bind to the light chain that extends the unconstrained length of the IgG hinge. As will be appreciated, the IgG2 and IgG4 hinge regions may be used in an unmodified form.

With respect to the choice of particular hinge regions for use in accordance with the present invention, we expect that each of the IgG hinge regions could function equivalently as a linker in our modified antibody design. Nevertheless, there are certain considerations that play a role upon the selection of the appropriate sequences to be utilized. For example, there is certain evidence that a longer hinge region may result in greater susceptibility to proteolysis Kim et al. Mol. Immunol. 32:467-475 (1995). If this result were to be observed, it will be appreciated that other hinge regions should be acceptable (i.e., IgG4 which has a relatively short hinge region). Further, it will be appreciated that such hinge regions may be modified to reduce, for instance, their length and/or their possibility for inter-disulfide bonds (i.e., removal of all cysteines from the molecule), or otherwise modify them so as to enhance their performance. Notwithstanding the foregoing, it should be reiterated that our interest resides in maximization of the half-life of the molecule and that simply because a molecule has the potential to be cleared more rapidly for one reason does not necessarily imply that its overall clearance rate will be drastically impacted.

As part of preliminary experiments to demonstrate that we were capable of generating cell lines secreting a modified Fc molecule, we selected a human gamma-1 sequence for the hinge. Thus, the modified molecule would comprise an IgG1 hinge coupled to a CH2-CH3 region as our initial FcRb binding domain to be conjugated to an IgG antibody. See FIG. 1. The gamma-1 hinge is the longest of the human gamma hinge regions and we anticipated this would allow for the most unconstrained linkage between the IgG antibody and the FcRb binding moieties. Although the gamma-1 hinge is the longest of the IgG hinge regions it also contains an additional cysteine capable of disulfide bond formation. In order to provide a less-reactive linker we decided to mutate this residue. In Table 1, the native IgG1 hinge structure is shown relative to the mutated form that was utilized:

TABLE 1

| | |
|---|---|
| Native IgG1 Hinge: | |
| Ala Glu Pro Lys Ser Cys Asp Lys Thr His Thr His Thr Cys | [SEQ ID:1] |
| Pro Pro | |
| Mutated IgG1 Hinge: | |
| Ala Glu Pro Lys Ser [Ser] Asp Lys Thr His Thr His Thr | [SEQ ID:2] |
| Cys Pro Pro | |

For the IgG antibody to which the FcRb binding moiety was to be bound was selected to be an IgG4 antibody with specificity to the lymphokine IL-8. The resulting modified antibody is linked at its carboxy terminus to a modified gamma-1 hinge (with the cysteine mutated to serine) which is further coupled to the gamma-1 CH2 and CH3 exons which contain the FcRb binding domain.

Additional constructs utilizing the same strategy will include shorter hinges corresponding to the other human gamma isotypes as are shown in Table 2:

TABLE 2

| | |
|---|---|
| Native IgG4 hinge: | |
| Glu Ser Lys Tyr Gly Pro Pro Cys Pro Ser Cys Pro | [SEQ ID:3] |
| Native IgG2 hinge: | |
| Glu Arg Lys Cys Cys Val Glu Cys Pro Pro Cys Pro | [SEQ ID:4] |

As will be appreciated, the present invention is principally focused upon extending the half-life of the molecule modified in accordance therewith. However, it will be further appreciated that, in accordance with the present invention, effector function can also be modified. Thus, FcRn binding moieties can also be designed to impart effector function. Using similar techniques as described herein, the effect of the additional FcRn binding moieties on the effector function of the different IgG isotypes can be imparted to molecules. For example, in accordance with the experiments described herein, the parent anti-IL-8 IgG4 antibody has relatively inactive effector function. Such molecule could be linked to other FcRn binding moieties that possess various effector functions. Similarly, parental antibodies that have active effector function (i.e., IgG1) can be modified with FcRn binding moieties to further enhance or augment or inhibit their effector function. For example, the linkage of a gamma-1 containing FcRn binding moiety to an antibody having a gamma-1 constant region might increase effector function by virtue of increased affinity or avidity, similar to what we have described for FcRb/FcRn binding. By a similar rationale, in connection with complement activation, multiple binding sites to the “ligand”, i.e., complement could lead to increased affinity or avidity between the modified molecule and its ligand and thus lead to greater effector function.

As will be appreciated, molecules designed and constructed in accordance with the invention can be readily tested for their ability to enhance in vivo half-life of the parental molecules. Methods of testing for these effects are described in detail in, for example, International Patent Application No. WO 97/43316 and U.S. Pat. No. 5,739,277, the disclosures of which are hereby incorporated by reference.

### EXAMPLES

The following examples, including the experiments conducted and results achieved are provided for illustrative purposes only and are not to be construed as limiting upon the present invention.

### Example 1

Generation of Antibodies

Antibodies for use in the present invention were prepared, selected, assayed, and characterized in accordance with the present Example.

Immunization and Hybridoma Generation:

The parental anti-IL-8 antibody utilized herein was generated as follows: XenoMouse Animals (8 to 10 weeks old) were immunized intraperitoneally with 25 mg of recombinant human IL-8 (Biosource International) emulsified in complete Freund's adjuvant for the primary immunization and in incomplete Freund's adjuvant for the additional immunizations carried out at two week intervals. This dose was repeated three times. Four days before fusion, the mice received a final injection of antigen in PBS. Spleen and lymph node lymphocytes from immunized mice were fused with the non-secretory myeloma NSO-bcl2 line (Ray and Diamond, 1994), and were subjected to HAT selection as previously described (Galfre and Milstein, 1981). A large panel of hybridomas all secreting IL-8 specific human IgG₂k which were thereafter cloned from the parental hybridoma and the heavy and light chain genes were placed into pee6.1 expression vectors and the heavy chain was recombinantly modified to result in expression on an IgG4.

ELISA Assay:

Antibodies generated as above were selected and detected as follows: ELISA for determination of antigen-specific antibodies in mouse serum and in hybridoma supernatants were carried out as described (Coligan et al., 1994) using recombinant human IL-8 to capture the antibodies. The concentration of human and mouse immunoglobulins were determined using the following capture antibodies: rabbit anti-human IgG (Southern Biotechnology, 6145-01), goat anti-human Igk (Vector Laboratories, AI-3060), mouse anti-human IgM (CGI/ATCC, HB-57), for human g, k, and m Ig, respectively, and goat anti-mouse IgG (Caltag, M 30100), goat anti-mouse Igk (Southern Biotechnology, 1050-01), goat anti-mouse IgM (Southern Biotechnology, 1020-01), and goat anti-mouse l (Southern Biotechnology, 1060-01) to capture mouse g, k, m, and l Ig, respectively. The detection antibodies used in ELISA experiments were goat anti-mouse IgG-HRP (Caltag, M-30107), goat anti-mouse Igk-HRP (Caltag, M 33007), mouse anti-human IgG2-HRP (Southern Biotechnology, 9070-05), mouse anti-human IgM-HRP (Southern Biotechnology, 9020-05), and goat anti-human kappa-biotin (Vector, BA-3060). Standards used for quantitation of human and mouse Ig were: human IgG₂ (Calbiochem, 400122), human IgMk (Cappel, 13000), human IgG₂k (Calbiochem, 400122), mouse IgGk (Cappel 55939), mouse IgMk (Sigma, M-3795), and mouse IgG₃l (Sigma, M-9019).

Determination of Affinity Constants of Fully Human Mabs by BIAcore:

Affinity measurement of purified human monoclonal antibodies, Fab fragments, or hybridoma supernatants by plasmon resonance was carried out using the BIAcore 2000 instrument, using general procedures outlined by the manufacturers.

Kinetic analysis of the antibodies was carried out using human IL-8 at 81 RU immobilized onto the sensor surface at a low density (1,000 RU correspond to about 1 ng/mm² of immobilized protein). The dissociation (kd) and association (ka) rates were determined using the software provided by the manufacturers, BIAevaluation 2.1.

Affinity Measurement by Radioimmunoassay:

¹²⁵I-labeled human IL-8 (1.5×10⁻¹¹ M or 3×10⁻¹¹ M) was incubated with purified anti-IL-8 human antibodies at varying concentrations (5×10⁻¹³ M to 4×10⁻⁹ M) in 200 ml of PBS with 0.5% BSA. After 15 hrs. incubation at room temperature, 20 ml of Protein A Sepharose CL-4B in PBS (1/1, v/v) was added to precipitate the antibody-antigen complex. After 2 hrs. incubation at 4° C., the antibody-¹²⁵I-IL-8 complex bound to Protein A Sepharose was separated from free ¹²⁵I-IL-8 by filtration using 96-well filtration plates (Millipore, Cat. No. MADVN65), collected into scintillation vials and counted. The concentration of bound and free antibodies was calculated and the binding affinity of the antibodies to the specific antigen was obtained using Scatchart analysis (2).

Receptor Binding Assays:

The IL-8 receptor binding assay was carried out with human neutrophils prepared either from freshly drawn blood or from buffy coats as described (Lusti-Marasimhan et al., 1995). Varying concentrations of antibodies were incubated with 0.23 nM [¹²⁵I]IL-8 (Amersham, IM-249) for 30 min at 4° C. in 96-well Multiscreen filter plates (Millipore, MADV N6550) pretreated with PBS binding buffer containing 0.1% bovine serum albumin and 0.02% NaN₃ at 25° C. for 2 hours. 4×10⁵ neutrophils were added to each well, and the plates were incubated for 90 min at 4° C. Cells were washed 5 times with 200 ml of ice-cold PBS, which was removed by aspiration. The filters were air-dried, added to scintillation fluid, and counted in a scintillation counter. The percentage of specifically bound [¹²⁵I]IL-8 was calculated as the mean cpm detected in the presence of antibody divided by cpm detected in the presence of buffer only.

Repertoire Analysis of Human Ig Transcripts Expressed in XenoMice and Their Derived Human Mabs:

Poly(A)⁺ mRNA was isolated from spleen and lymph nodes of unimmunized and immunized XenoMice using a Fast-Track kit (Invitrogen). The generation of random primed cDNA was followed by PCR. Human V_{H} or human Vk family specific variable region primers (Marks et. al., 1991) or a universal human V_{H} primer, MG-30 (CAGGTGCAGCTGGAGCAGTCIGG) was used in conjunction with primers specific for the human Cm (hmP2) or Ck (hkP2) constant regions as previously described (Green et al., 1994), or the human g2 constant region MG-40d; 5′-GCTGAGGGAGTAGAGTCCTGAGGA-3′. PCR products were cloned into pCRII using a TA cloning kit (Invitrogen) and both strands were sequenced using Prism dye-terminator sequencing kits and an ABI 377 sequencing machine. Sequences of human Mabs-derived heavy and kappa chain transcripts were obtained by direct sequencing of PCR products generated from poly(A⁺) RNA using the primers described above. All sequences were analyzed by alignments to the “V BASE sequence directory” (Tomlinson et al., MRC Centre for Protein Engineering, Cambridge, UK) using MacVector and Geneworks software programs.

Preparation and Purification of Antibody Fab Fragments:

Antibody Fab fragments were produced by using immobilized papain (Pierce). The Fab fragments were purified with a two step chromatographic scheme: HiTrap (Bio-Rad) Protein A column to capture Fc fragments and any undigested antibody, followed by elution of the Fab fragments retained in the flow-through on strong cation exchange column (PerSeptive Biosystems), with a linear salt gradient to 0.5 M NaCl. Fab fragments were characterized by SDS-PAGE and MALDI-TOF MS under reducing and non-reducing conditions, demonstarting the expected ^{˜}50 kD unreduced fragment and ^{˜}25 kDa reduced doublet. This result demonstrates the intact light chain and the cleaved heavy chain. MS under reducing conditions permitted the unambiguous identification of both the light and cleaved heavy chains since the light chain mass can be precisely determined by reducing the whole undigested antibody.]

### Example 2

Cloning IL-8 Specific Parent Antibody Genes

In order to isolate the antibody genes of the parent anti-IL-8 antibody, we cloned genes encoding the heavy and light chain fragments out of a selected hybridoma cell line, D1.1 encoding and secreting the antibody. Gene cloning and sequencing was accomplished as follows:

Poly(A)⁺ mRNA was isolated from approximately 2×10⁵ hybridoma cells derived from immunized XenoMice using a Fast-Track kit (Invitrogen). The generation of random primed cDNA was followed by PCR. Cloning was done utilizing primers unique to 5′ untranslated region of VH and VK gene segments and the appropriate 3′ primers using standard molecular biology techniques. Each chain was placed independently into a standard CMV promoter driven expression vector. The heavy chain was cloned in a manner such that the heavy chain would contain the human gamma 4 constant region.

### Example 3

Generation of the FcRn Binding Moiety

In order to generate the modified antibodies in accordance with the invention, we next prepared a FcRn binding moiety through cloning out and modification of the selected FC genes followed by cloning to the parental anti-IL-8 heavy chain gene. This procedure was accomplished as follows:

The strategy used to construct antibody modified with the FcRn binding moiety is depicted in FIG. 2.

In connection with the strategy, we first decided to introduce a unique restriction site into the 3′ terminus of the gamma-4 constant region so as to assist with the linking the antibody with the FcRn binding moiety. To this end, without introducing any amino acid changes we introduced a new restriction site (Bsu36I) in the 3′ terminus of the gamma-4 constant region. The process is depicted in FIG. 2.

In step 1 on FIG. 2, the nucleotide sequence encoding the last 4 amino acids in the native and modified form are shown. Specific primer sequences, utilized in PCR, to accomplish this change are shown in Step 3. Primer 1 contains a Dra III site within the gamma-4 CH3 exon and primer 2 introduces the Bsu36I site. Primer 3 also contains a Bsu36I site as well as sequences homologous to the human gamma 1 hinge region. Primer 3 also includes nucleotide changes that convert the cysteine to a serine in the gamma 1 hinge. Primer 4 is complementary to the 3′ terminus of the gamma lgene (3′ flanking sequences) and includes an EcoRI site for cloning. The parent VDJ-gamma4 vector is digested with DraIII and EcoRI. The amplified products of primer 1 and primer 2 are digested with DraIII and Bsu36I and the amplification product of the gamma-1 sequence with primer 3 and primer 4 are digested with Bsu36I and EcoRI; a three way ligation of the two digested PCR products and the vector (DraIII-Bsu36I-EcoRI) generate the modified antibody construct. The resulting construct has the complete IgG4 antibody linked to FcRn binding moiety as shown in FIG. 1.

As will be appreciated, where other gamma-constant region genes are utilized, slightly different but similar procedures can be utilized for linking the molecules. For example, the 5′g1 oligo would be replaced with hinge sequences corresponding to the different IgG isotypes. The primer would be slightly longer to encode the 12 amino acids of the hinge as well as 10 nucleotides of the IgG1 CH2 sequence. This strategy will allow any hinge sequence to link the IgG4 and IgG1 FcRp binding domains.

### Example 4

Expression and Analysis of the Structure of the Modified Antibody

In order to generate sufficient amounts of material for in vitro and in vivo studies, stable cell lines secreting the modified antibodies were generated. The use of the NSO myeloma to generate stable cell lines allows material to be purified from both culture supernatants as well as from ascites. In order to confirm the structure of the above-modified antibody construct, restriction digests and DNA sequencing was performed. The analysis of the protein, described below, was facilitated by the design of the construct so that it contains two different IgG isotypes on the same molecule.

Cell lines can be generated through any number of conventional methods. In one example, we generated NSO myeloma cell lines expressing the modified antibody constructs by co-transfecting the modified heavy chain and a plasmid containing the puromycin selectable marker into a NSO cell line that had previously been generated to stably express the human kappa light chain found in the parent hybridoma. Standard electroporation and puromycin selection protocols were followed to generate cell lines expressing fully assembled modified heavy chain and human kappa light chain antibodies. The cell lines that were generated express the modified antibody at levels of about 200 ng/ml. Current levels of expression allow us to generate sufficient materials for our in vitro and in vivo studies with approximately 1 liter of cell culture supernatants. Production of ascites from these clones can also be accomplished.

The modified antibodies secreted by the cell lines can be purified using a number conventional techniques. In one example, we purify such antibodies through use of protein A column purification techniques. Because we cannot predict the purification of the modified antibody (it will have two potential protein A binding sites) it is also useful to utilize alternative chromatographic matrices including protein K and anti-IgG columns for purification, either alone or in combination with protein A purification and or the others. In addition, as will be appreciated, it is possible to further modify the antibody to facilitate the purification.

Following purification, a number of assays may be performed to confirm the structure of the modified antibody protein. In one example, we utilized an ELISA sandwich assay to confirm the existence of the additional FcRn binding domain. In the assay, standard ELISA plates (Nunc immunoplates) were coated with an IgG1 specific antibody (cat # calbiochem 411428#), as a capture antibody, and detection was carried out with an HRP conjugated mouse anti-IgG4 (cat #southern biotech 9200-05) as the secondary antibody. The ELISA results (not shown) demonstrate that the molecule can be specifically captured for human IgG1 and detected with anti-human IgG4. Antigen specific ELISAs to IL-8 were also performed to confirm that the presence of an additional FcRb binding domain has not altered the antigen binding specificity of the parent antibody (data not shown).

We also analyzed the modified antibodies using PAGE gels and western blots in order to confirm the increased size (which should be, and was, approximately 26 kd higher in weight than the unmodified antibody. The result was the production of an approximately 76 kd protein instead of a 50 kd protein. In certain experiments, there was also a lower molecular weight species present at 54 kd that could be a proteolytic product. In addition, under non-reducing conditions, using a human IgG1 specific antibody, we observed a protein product with a molecular weight of approxiamately 200 kd. (data not shown).

Accordingly, the modified antibodies in accordance with the invention appear to have the predicted structure. The modified antibody recognizes the specific antigen to which the VDJ-region of the parent antibody was specific, it has the predicted molecular weight, and contains both the IgG4 and IgG1 constant regions. In addition, because the binding of protein A has been shown to involve the same region as FcRb binding Raghavan et al. Immunity 1:303-315 (1994), binding studies with protein A can also be used to indirectly confirm that the FcRb binding domain of the modified antibody is correctly folded and functional. It is also possible to to use I 125—Protein A in a binding assay to determine if the modified antibody is binding to two protein A molecules simultaneously. Similarly, a BIAcore experiment with protein A can also be used to determine if the second binding site for a ligand in the modified antibody molecule increases the affinity to the ligand. Further confirmation of the binding of the modified antibody molecules in accordance with the invention are discussed below in connection with the in vivo binding studies that are described below.

### Example 5

Receptor Binding Studies

In order to study the binding affinities of the modified antibodies to the FcRb receptor, purified FcRb receptor is required. Cloning and expression of the FcRb for binding studies will be carried out essentially as previously described (Vaughn and Bjorkman 1997, Raghaven et al 1995a, and Raghaven et al 1995b, Raghaven et al 1994, Ghetie). For BIAcore studies, a secreted form of the human FcRn (a heterodimer composed of residues 1-269 of the FcRp heavy chain associated with the b2 microglobulin) will be generated. The FcRn will also include a polyhistidine (His 6×) tag at the carboxy terminus of the FcRp heavy chain in order to facilitate screening, purification as well as, potentially, the immobilization of FcRp to the BIAcore chip. RT-PCR of human placental RNA (Strategene) will be used to generate the appropriate cDNAs that will be cloned into standard mammalian expression vectors and subsequently co-transfected into CHO cells. Clones secreting the truncated FcRb heterodimer will be identified using a sandwich ELISA. Plates will be coated with human IgG and an anti-His secondary antibody will be used for detection (Qiagen). The highest expressers will be expanded and the secreted FcRp will be purified using pH-dependent binding to a rat IgG column (Gastinel et al 1992). If additional purification is required, a standard nickel based matrix will be used to take advantage of the His-tag.

We will also generate a second vector that expresses a lipid linked beta-2-microglobulin (B2m) protein that has previously been utilized for FcRb cell binding studies (Gastinel et al. 1992 and Raghaven et al 1994). The lipid linked B2m contains the phosphatidylinositol-anchoring signal of DAF (residues 311-347) linked to its carboxy terminal amino acid. Cell lines that express FcRp in a stable manner on their surfaces, will be generated by co-transfecting the truncated FcRb heavy chain along with the lipid-linked B2m. Each expression vector will carry a distinct selectable marker (i.e. hygromycin and puromycin) so that double selection can be performed. Cell lines that express the FcRp on their cell surface in a stable manner will be identified by incubating the cells at pH 6.0 with a FITC conjugated human IgG followed by analysis on FACS. Subsequent FACS analysis at both pH 6.0 and pH 7.4 will confirm that the binding is mediated by FcRp. High expressers will be identified by their fluorescent intensity and sorted.

In addition to generating recombinant cell lines that express FcRp on their surface we will also perform binding assays with brush-border membranes isolated from newborn rats. Isolation of brush-border membranes will be carried according to the modified method described by Wallace and Reese 1980. Suckling rats (9-14 days old) will be killed by cervical dislocation (see section F) and the proximal half of the jejunum will be removed into ice-cold 5 mM-EDTA, pH 7.4 containing PMSF (2 ug/ml) and pepstatin (1 ug/ml) as proteinase inhibitors. The protocol shown below will be followed to isolated cells appropriate for binding assays.

The sequence and cloning of the FcRb has been described previously (Raghavan et al. PNAS 92: 11200-11204, 1995; Kim et al. Eur. J. Immunol. 24: 2429-2434, 1994; Raghavan et al. Immunity 1: 303-315, 1994; Vaughn and Bjorkman Structure 663-73, 1998; Vaughn and Bjorkman Biochemistry 36: 9374-9380, 1997) and we will follow the published protocols for generating the FcRb receptor for both BIAcore and cell binding assays.

8 step procedure for the isolation of brush-border membranes form the neonatal rat small intestine (Wallace and Reese Biochem J 188: 9-16 (1980):

Intestinal mucosa, from proximal half of small intestine of 3-5 rats, scraped into 50 ml of 5 mM-EDTA, pH 7.4.

Scrapings repeatedly drawn into Pasteur pipette until a uniform opaque cream-yellow suspension is obtained (all muscle fragments removed)

Hyaluronidase added, as a 10 mg/ml solution in 5 mM-EDTA, pH 7.4, to a final concentration of 0.5 mg/ml; mixture swirled repeatedly at room temperature for 30 minutes.

Suspension forced through a 23-gauge needle

Suspension centrifuged at 100 g for 20 min at 4 C. and the supernatant discarded

Pellet is resuspended in a small volume (1-3 ml) of 90 mM NaCl/0.8 mM-EDTA, pH 7.4, containing deoxyribonuclease 1 (0.2 mg/ml); left at room temperature for 10 minutes

Repeat step 5

Pellet resuspended in assay buffer pH 6.0 and protein concentration (Bio-Rad)

Affinity constants (Ka) for the binding of modified and unmodified antibodies will be determined by the direct competition method. I¹²⁵ labeled antibody (Amersham) will be added at a final concentration of 0.5 nM to 190 ug of membrane protein or 5×10⁵ cells. Triplicate assays with labeled IgG (or modified IgG), different concentrations of unlabeled IgG and binding buffer (pH 6.0) will be performed in a total volume of 0.5 ml. Samples will be incubated in a shaking incubator at 37 C. for 2 hour. After incubation the sample will be centrifuged at 2000 g for 10 minutes and washed three times in cold MES-BSA buffer. The amount of protein non-specifically bound will be determined by measuring the radioactivity after an additional washing in 50 mM phosphate buffer pH 7.4 which will specifically release the bound FcRp. The data will be analyzed by the method of Scatchard (1949). The parameters of the Scatchard equation (Ka and n) will be evaluated by using a computed least-squares fit according to the method of Klotz and Hunston (1971).

Competition experiments will also be performed, by allowing the labeled IgG (or modified IgG), 0.5 nM, to come to equilibrium and then diluting the membrane pellet at least 10 volumes in the presence and absence of unlabeled IgG (10 mM). At appropriate time intervals (1, 2, 5, 10, 20, 30,40, 50, 60, 70, 80, and 90 minutes) samples will be removed and layered on to ice-cold buffer containing a BSA solution (22 mg/ml) in sealed off Pasteur pipettes. These will be centrifuged at 100× g for 20 minutes at 4° C. The samples will then be frozen and the tips of the pipettes containing the pellet will be broken off and radioactivity of both the pellet and frozen supernatant will be counted. The rate constant will be determined from first-order rate plots of the data.

The rate constant will be determined from first-order rate plots of the data.

### Example 6

In Vitro Binding Studies Using BIAcore

Kinetic studies of FcRp and the modified IgGs will be conducted utilizing the purified soluble FcRp described above and the BIAcore 2000 biosensor system (BIAcore, Inc). Previous work demonstrated that in order to achieve high-affinity binding on the biacore comparable to that observed on the cell surface, the receptor, FcRp and not the IgG ligand, must be immobilized on the biosensor surface (Vaughn and Bjorkman 1997). It is hypothesized that the immobilization of FcRp is more representative of the physiologically constrained conditions of an integral membrane protein. The conditions for studying Ig and FcRp interactions have been described previously (Raghavan et al 1994 and 1995) and essentially involves immobilizing soluble FcRp to dextran coated gold surface using standard amine coupling chemistry as described in the BIAcore manual. The kinetic data of the interaction will be analyzed using BIAevalution 3.0 software that uses global fitting anlaysis that permit simultaneous fitting of all the curves in the working set, with a simultaneous fitting for the association and dissociation phases of the interaction. The expected value for the high affinity interaction of an unmodified IgG to FcRp is in the range of 17 to 93 nM (Vaughn and Bjorkman 1997).

### Example 7

In Vitro Half-Life Determination Through Protein A Binding Assay

Human anti IL-8 IgG4 was modified to contain an additional Fc domain comprising the hinge-CH2-CH3 region as described above. Since protein A and the FcRb were shown to bind to overlapping sites on the IgG molecule we also speculated that the modified antibody would also have an increased affinity for protein A.

In order to determine if the modified antibody has a higher affinity for protein A than the parental antibody, we developed an in vitro assay to measure protein A binding. We compared the affinity of the 39.7, the unmodified parental anti IL-8 IgG4 (single Fc-Ig heavy chain) and the modified antibody FcRb (2Fc-Ig heavy chain). Using equivalent amount of antibody (as determined by ELISA) we looked at binding to protein A in increasing amounts of IgG competitor. The competitor IgG because it has an unmodified constant domain was anticipated to bind to protein A with the same affinity as 39.7 (single binding site). The method involved mixing a constant amount of the anti IL-8 antibodies with varying amounts of irrelevant IgG competitor (one that does not bind to Il-8). Protein A conjugated to horseradish peroxidase (HRP) was added and binding was allowed to proceed in solution. Protein A binding was determined by an ELISA based assay using IL-8 coated plates.

Experiment 1: Serial Dilution Analysis to Determine Optimal Reagent Concentrations.

Serial dilution was preformed to determine optimal antibody and protein A concentrations to be used in the subsequent ELISA analysis. In this protocol human recombinant IL-8 (Biosource, Foster-City Calif.) was used as a solid phase coating reagent at 0.5 mg/ml. The sample antibody, human anti IL-8 antibody 39.7 or the modified antibody, at 1 mg was incubated with different concentrations of HRP conjugated protein A (0.1 to 1 mg) for 1 hr at room temperature. Serial dilutions of the different mixes were dispensed onto the IL-8 coated plate. Absorption results confirmed that 1 mg of protein A binds 5 mg of human IgG and our following experiments were performed at antibody-protein A ratio of 1:10.

Experiment 2: Inhibition of Protein A Binding by a Competitor.

The same protocol described above was utilized incubating the 1 mg of anti IL-8 antibodies with different concentrations of IgG1 competitor antibody. The competitor, 0.5 mg up to 8 mg, was added followed by the addition of 100 ng of HRP conjugated Protein A. Serial dilutions of the different mixes were dispensed onto the IL-8 coated plate. Absorption results showed that:

1. There was no difference in protein A binding between the modified and normal antibodies. Equivalent molar amounts of the normal and the modified antibody bind protein A at the same ratio (1:1).

2. The modified antibody was less sensitive to competitor than the parental antibody. Approximately twice as much competitor antibody was required to reduce the binding of the modified antibody to the same levels as the parental antibody. We believe this preliminary result supports our hypothesis that the additional FcRb binding domain is able to increase the affinity (on rate) for binding to protein A.

### Example 8

In Vivo Half-Life Determination

In addition to in vitro binding studies, the most important criteria is weather the modified antibodies do in fact have a longer serum half-lives. The use of a mouse system to study human antibody pharmokinetics is available for this purpose, Junghans and Anderson PNAS 93: 5512-5516 (1996). The kinetic studies to test the modified molecules can be done in mice, because human IgG Fc interact just as well as mouse Fc do with the mouse FcRB receptor (Artandi et al PNAS 89:94-98 (1992); Fahey and Robinson, A. G. J Exp. Med 118: 845-868 (1963). The method that will be used to study the half-lives of modified antibodies in accordance with the invention can be accomplished through use of a variety of techniques. In one example, the following antibodies will be assayed 1) the parent IgG4 antibody, 2) a human IgG1 antibody as a control and 3) the modified antibody described above. Each of these molecules will be iodinated and thereafter injected into mice as described below using the procedures described in Junghans and Anderson PNAS USA 93:5512-5516 (1996). The protection receptor for IgG catabolism is the b2-microglobulin-containing neonatal intestinal transport receptor. Junghans and Waldmann J. Exp. Med 183, 1587-1602 (1996). Such procedures are outlined below:

As will be appreciated, all human IgG's have the same survival kinetics excepting IgG3 [Waldman and Strober Progr Allergy 13: 1-110, (1969) ], which is less well protected by FcRp due to alterations in the FcRb binding site [Burmeister et al Nature 372: 379-83 (1994)].

All in vivo data will be analyzed by two-compartment pharmacokinetic models to derive catabolic rate constants, beta phase rate constants, mean residence time, and other measures. To rule out biosynthetic anomalies, samples will initially be “screened” in recipient animals to remove aggregated or poorly folded protein. Two sets of animals will be employed: wild-type animals which have normal FcRB expression and animals which are knocked out for FcRB function by the b2m-/-genotype [Junghans and Anderson PNAS: 93: 5512-6 (1996)]. In the wildtype animals, we predict that the presence of the FcRB will allow discrimination of normal Fc and Fc2 IgG molecules, with prolonged survival of the latter. An increased survival of greater than two-fold will indicate higher than monovalent binding of Fc2 to receptor. In the knockout animals that lack functional FcRB, all molecules should exhibit equal, accelerated survival times expected of unprotected plasma proteins [Junghans and Anderson PNAS: 93: 5512-6 (1996); Junghans Immunol Res 16: 29-57. (1997)].

The following is an outline of the experiments:

Protein Labeling

20-100 mcg of Protein (IgG1, IgG4, IgG-Fc2) human IgG (Gammimmune, Cutter)

Iodination (I125 or I131) with iodobeads (Pierce) to specific activity of 1-3 mcCi/mcg.

“Screening” of Labeled, Biosynthetic Antibody

This is done in analogy to McFarlane and others [McFarlane Ann NY Acad Sci 70: 19-25 (1957); Pollock et al. Eur J Immunol. 20: 2021-27 (1990)], which removes improperly folded or denatured proteins before they are injected, which otherwise confound the pharmocokinetics analysis. 1 ml of each labeled protein for pharmacokinetics is injected i.p. into a mouse. The mice are exsanguinated under anesthesia after 48 hours. The blood is processed to serum and characterized for recovery of radioactive protein. This screened protein is used for the further studies.

Preliminary Tests of Labeled and “Screened” Proteins

Prior to conducting the following, large scale tests, we will perform small scale labeling, with screening of a portion of the labeled materials, and compare pharmacokinetics of screened and unscreened portions of the labeled proteins. This will be done to ascertain the relative biologic intactness of the native and Fc2 molecules, by this biologic criterion. It will also establish the parameters to expect in the following, definitive studies.

Wildtype C57BL6/J mice will be utilized in this set of experiments.

3 mice for screening (one for each antibody)

12 mice for pharmacokinetics (two mice each, for each antibody, +/−screened)

For three sets of protein, this requires 15 mice. Allowing for a potential repeat of the study, this requires 30 mice.

Testing Prolongation of Survival of Modified Antibodies

Animal facility-raised mice in “clean” facilities have low IgG levels relative to feral mice due to reduced pathogen exposure [Sell and Fahey J. Immunol 93:81-7 (1964)]. To create higher IgG levels, to generate the competition for receptor, bulk IgG is administered to raise the plasma IgG levels, as we did previously [Junghans and Anderson PNAS: 93: 5512-6 (1996)]. Human IgG binds to the murine FcRB similar to mouse IgG and competes for receptor binding [Fahey and Robinson J Exp Med 118:845-68 (1963)]. Accordingly, bulk human gamma globulin is tracer labeled with I¹²⁵ to allow quantitation of plasma levels of administered human bulk IgG. Endogeneous mouse IgG levels are measured by ELISA, and added to the human IgG levels to yield a total concentration of IgG [Junghans and Anderson PNAS: 93: 5512-6 (1996)].

Wildtype C57BL6/J mice are used in this set of experiments. Five sets of 5 mice each are employed, with different doses of I¹²⁵ bulk IgG to generate five groups of mice differing in plasma IgG levels. Mice are subsequently bolus-injected with radiolabeled I131 antibodies by tail vein. Blood samples are collected over a period of 5-8 days and analyzed by pharmacokinetic models to derive survival t1/2 values. These are plotted versus plasma concentrations of total IgG. Our hypothesis of greater affinity and resistance to catabolism predicts survival t1/2 values that show progressive advantage for the 2Fc molecules as higher IgG levels generate competition with the I131 labeled IgG proteins.

For three sets of proteins, this requires 75 mice. Allowing for a potential repeat of the study, this requires 150 mice.

Testing Role of FcRB in Prolongation of Survival.

Wildtype and FcRB−/− mice are studied for relative survival of each protein under two conditions, with no added bulk IgG and with a high dose of added bulk IgG. If FcRB regulates the advantage of survival of the Fc2 IgG, then that advantage should disappear in the absence of FCRB, showing equal, accelerated survival of the normal Fc and Fc2 IgGs.

Four sets of 5 mice for each IgG (high and low IgG, wiltype and knockout). For three sets of proteins, this requires 60 mice. Allowing for potential repeat of the study, this requires 120 mice.

The end point of this study includes the affinity measurements determined by binding studies on cells and the BIAcore and the half-life calculations and characteristics determined from the in vivo studies. The criteria that we have set for considering applying for continuation into a phase 2 study would require an modified antibody to have at least a 50% longer half-life than the parent antibody, ie from 3 days to 4.5 days in mice. Extrapolating to humans this would correspond to a half-life from typically around 23 days for a standard antibody to 30 days for the modified antibody.

### INCORPORATION BY REFERENCE

All references cited herein, including patents, patent applications, papers, text books, and the like, and the references cited therein, to the extent that they are not already, are hereby incorporated herein by reference in their entirety. In addition, the following references are also incorporated by reference herein in their entirety, including the references cited in such references:

Abertsen et al., “Construction and characterization of a yeast artificial chromosome library containing seven haploid human genome equivalents.” Proc. Natl. Acad. Sci. 87:4256 (1990).

Anand et al., “Construction of yeast artificial chromosome libraries with large inserts using fractionation by pulsed-field gel electrophoresis.” Nucl. Acids Res. 17:3425-3433 (1989).

Berman et al. “Content and organization of the human Ig V_{H} locus: definition of three new V_{H} families and linkage to the Ig C_{H} locus.” EMBO J. 7:727-738 (1988).

Brezinschek et al., “Analysis of the heavy chain repertoire of human peripheral B-cells using single-cell polymerase chain reaction.” J. Immunol. 155:190-202 (1995).

Brownstein et al., “Isolation of single-copy human genes from a library of yeast artificial chromosome clones.” Science 244:1348-1351 (1989).

Bruggeman et al. PNAS USA 86:6709-6713 (1989).

Bruggemann et al., “Human antibody production in transgenic mice: expression from 100 kb of the human IgH locus.” Eur. J. Immunol. 21:1323-1326 (1991).

Bruggeman, M. and Neuberger, M. S. in Methods: A companion to Methods in Enzymology 2:159-165 (Lerner et al. eds. Academic Press (1991)).

Bruggemann, M. and Neuberger, M. S. “Strategies for expressing human antibody repertoires in transgenic mice.” Immunology Today 17:391-397 (1996).

Chen et al. “Immunoglobulin gene rearrangement in B-cell deficient mice generated by targeted deletion of the J_{H} locus” International Immunology 5:647-656 (1993)

Choi et al. “Transgenic mice containing a human heavy chain immunoglobulin gene fragment cloned in a yeast artificial chromosome” Nature Genetics 4:117-123 (1993)

Coligan et al., Unit 2.1, “Enzyme-linked immunosorbent assays,” in Current protocols in irnmunology (1994).

Cook, G. P. and Tomlinson, I. M., “The human immunoglobulin V_{H} repertoire.” Immunology Today 16:237-242 (1995).

Cox et al., “A directory of human germ-line Vx segments reveals a strong bias in their usage.” Eur. J. Immunol. 24:827-836 (1994).

Dariavach et al., “The mouse IgH 3′-enhancer.” Eur. J. Immunol. 21:1499-1504 (1991).

Den Dunnen et al., “Reconstruction of the 2.4 Mb human DMD-gene by homologous YAC recombination.” Human Molecular Genetics 1:19-28 (1992).

Feeney, A. J. “Lack of N regions in fetal and neonatal mouse immunoglobulin V-D-J junctional sequences.” J. Exp. Med. 172:137-1390 (1990).

Fishwild et al., “High-avidity human IgGkmonoclonal antibodies from a novel strain of minilocus transgenic mice.” Nature Biotech. 14:845-851 (1996).

Flanagan, J. G. and Rabbitts, T. H., “Arrangement of human immunoglobulin heavy chain constant region genes implies evolutionary duplication of a segment containing g, e, and a genes.” Nature 300:709-713 (1982).

Galfre, G. and Milstein, C., “Preparation of monoclonal antibodies: strategies and procedures.” Methods Enzymol. 73:3-46 (1981).

Gemmill et al., “Protocols for pulsed field gel electrophoresis: Separation and detection of large DNA molecules.” Advances in Genome Biology 1:217-251 (1991).

Gill et al., “Monoclonal anti-epidermal growth factor receptor antibodies which are inhibitors of epidermal growth factor binding and antagonists of epidermal growth factorstimulated tyrosine protein kinase activity.” J. Biol. Chem. 259:7755 (1984).

Green et al., “Antigen-specific human monoclonal antibodies from mice engineered with human Ig heavy and light chain YACs.” Nature Genetics 7:13-21 (1994).

Hermanson et al., “Rescue of end fragments of yeast artificial chromosomes by homologous recombination in yeast.” Nucleic Acids Res. 19:4943-4948 (1991).

Huber et al., “The human immunoglobulin k locus. Characterization of the partially duplicated L regions.” Eur. J. Immunol. 23:2860-2967 (1993).

Jakobovits, A., “Humanizing the mouse genome.” Current Biology 4:761-763 (1994).

Jakobovits, A., “Production of fully human antibodies by transgenic mice.” Current Opinion in Biotechnology 6:561-566 (1995).

Jakobovits et al., “Germ-line transmission and expression of a human-derived yeast artificial-chromosome.” Nature 362:255-258 (1993).

Jakobovits, A. et al., “Analysis of homozygous mutant chimeric mice: Deletion of the immunoglobulin heavy-chain joining region blocks B-cell development and antibody production.” Proc. Natl. Acad. Sci. USA 90:2551-2555 (1993).

Kawamoto et al., “Growth stimulation of A431 cells by epidermal growth factor: Identification of high affinity receptors for EGF by an anti-receptor monoclonal antibody.” Proc. Nat. Acad. Sci., USA 80:1337-1341 (1983).

Lonberg et al., “Antigen-specific human antibodies from mice comprising four distinct genetic modifications.” Nature 368:856-859 (1994).

Lusti-Marasimhan et al., “Mutation of Leu25 and Val27 introduces CC chemokine activity into interleukin-8.” J. Biol. Chem. 270:2716-2721 (1995).

Marks et al., “Oligonucleotide primers for polymerase chain reaction amplification of human immunoglobulin variable genes and design of family-specific oligonucleotide probes.” Eur. J. Immunol. 21:985-991 (1991).

Matsuda et al., “Structure and physical map of 64 variable segments in the 3′ 0.8-megabase region of the human immunoglobulin heavy-chain locus.” Nature Genetics 3:88-94 (1993).

Max, E. Molecular genetics of immunoglobulins. Fundamental Immunology. 315-382 (Paul, WE, ed., New York: Raven Press (1993)).

Mendez et al., “A set of YAC targeting vectors for the interconversion of centric and acentric arms.” Cold Spring Harbor Laboratory Press, Genome Mapping and Sequencing meeting, 163 (1993).

Mendez et al., “Analysis of the structural integrity of YACs comprising human immunoglobulin genes in yeast and in embryonic stem cells.” Genomics 26:294-307 (1995).

Ray, S. and Diamond, B., “Generation of a fusion partner to sample the repertoire of Splenic B-cells destined for apoptosis.” Proc. Natl. Acad. Sci. USA 91:5548-5551 (1994).

Sato et al., “Biological effects in vitro of monoclonal antibodies to human epidermal growth factor receptors” Mol. Biol. Med. 1:511-529 (1983).

Schiestl, R. H. and Gietz, R. D., “High efficiency transformation of intact yeast cells using stranded nucleic acids as a carrier.” Curr. Genet. 16:339-346 (1989).

Sherman et al., “Laboratory Course Manual for Methods in Yeast Genetics.” (Cold Spring Harbor Laboratory Press, Cold Spring Harbor, N.Y. (1986)).

Silverman et al., “Meiotic recombination between yeast artificial chromosomes yields a single clone containing the entire BCL2 protooncogene.” Proc. Natl. Acad. Sci. USA 87:9913-9917 (19______).

Srivastava, A. and Schlessinger, D., “Vectors for inserting selectable markers in vector arms and human DNA inserts of yeast artificial chromosomes (YACs).” Gene 103:53-59 (1991).

Taylor et al., “A transgenic mouse that expresses a diversity of human sequence heavy and light chain immunoglobulins.” Nucleic Acids Research 20:6287-6295 (1992).

Taylor et al., “Human immunoglobulin transgenes undergo rearrangement, somatic mutation and class switching in mice that lack endogenous IgM.” International Irnmunology 6:579-591 (1994).

Tuaillon et al., “Human immunoglobulin heavy-chain minilocus recombination in transgenic mice: gene-segment use in m and g transcripts.” Proc. Natl. Acad. Sci. USA 90:3720-3724 (1993).

Tuaillon et al. “Analysis of direct and inverted DJ_{H} rearrangements in a human Ig heavy chain transgenic minilocus” J. Immunol. 154:6453-6465 (1995)

Vaughan et al., “Human antibodies with subnanomolar affinities isolated from a large non-irnmunized phage display library.” Nature Biotech. 14:309-314 (1996).

Wagner et al., “The diversity of antigen-specific monoclonal antibodies from transgenic mice bearing human immunoglobulin gene miniloci.” Eur. J. Irnmunol. 24 :2672-2681 (1994).

Weichhold et al., “The human immunoglobulin k locus consists of two copies that are organized in opposite polarity.” Genomics 16:503-511 (1993).

Yamada, M. et al., “Preferential utilization of specific immunoglobulin heavy chain diversity and joining segments in adult human peripheral blood B lymphocytes.” J. Exp. Med. 173:395-407 (1991).

Artandi et al. Proc Natl Acac Sci USA 89:94-98 (1992)

Burmeister et al. Nature 372:379-383 (1994)

Fahey and Robinson J Exp Med 118:845-868 (1963)

Ghetie and Ward Immunol Today 18:592-598 (1997)

Ghetie et al. Nature Biotechnology 15:637 (1997)

Godfrey Adademic Press 7-11 (1983)

Junghans Immunol Res 16:29-57 (1997)

Junghans Immunol Today (1998)

Junghans and Anderson Proc Natl Acad Sci USA 93:5512-5516 (1996)

Junghans et al. Proc Natl Acac Sci USA 93:5512-5516 (1996)

Kim et al. Eur J Immunol 24:542-548 (1994)

Kim et al. Scand J Immunol 40:457-465 (1994)

Kim et al. Mol Immunol 32:467-475 (1995)

Mason and Williams Biocyhem J 187:1-20 (1980)

McFarlane Ann NY Acad Sci 70:19-25 (1957)

Medesan et al. J Immunol 158:2211-2217 (1997)

Medesan et al. Eur J Immunol 26:2533-2536 (1996)

Mendez et al. Nature Genetics 15:146 (1997)

Pollock et al. Eur J Immunol 20:2021-2027 (1990)

Raghavan et al. Biochemistry 34:14649-14657 (1995)

Raghavan et al. Immunity 1:303-315 (1994)

Raghavan et al. Proc Natl Acac Sci USA 92:11200-11204 (1995)

Segal et al. Mol Immunol 20:1177-1189 (1983)

Sell and Fahey J Immunol 93:81-87 (1964)

Vaughn and Bjorkman Biochemistry 36:9374-9380 (1997)

Vaughn and Bjorkman Stucture 6:63-73 (1998)

Waldmann and Jones Protein Turnover 9:5-23 (1973)

Waldmann and Strober Progr Allergy 13:1-110 (1969)

Wallace and Rees Biochem J 188:9-16 (1980)

Equivalents

The foregoing description and Examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

We claim:

1. A method of modifying the half life of an antibody having a first FcRn binding domain, comprising: physically linking said antibody to a second FcRn binding domain.

2. The method of claim 1, wherein said physical linking is performed by recombinantly engineering the nucleic acid that encodes said antibody.

3. A modified antibody, said antibody comprising at least a first and second FcRn binding domain.

4. The antibody of claim 3, wherein said antibody has a serum half-life in mammals greater than said antibody lacking said second FcRn binding domain.

5. The antibody of either claim 3 or claim 4, wherein said antibody binds specifically to IL-8.

6. An antibody produced by the process of claim 1.

7. A modified antibody molecule comprising an exogenous FcRn binding domain physically linked to a constant region domain of the antibody.

8. The modified antibody of claim 7, wherein the antibody is a single chain antibody.

9. The modified antibody of claim 7, wherein the antibody is a dimer.

10. The modified antibody of claim 7, wherein the antibody comprises an IgG heavy chain.

11. The modified antibody of claim 7, wherein the antibody comprises and IgM heavy chain.
